(19) United States
(12) Patent Application Publication
(10) Pub. No.: US 2024/0173301 A1
(43) Pub. Date: May 30, 2024

(54) 2-IMINOBIOTIN FOR USE IN THE TREATMENT OF STROKE

(71) Applicant: NEUROPHYXIA B.V., 's-Hertogenbosch (NL)

(72) Inventors: Ido Remy VAN DEN WIJNGAARD, Hertogenbosch (NL); Cacha Marie Pétronelle Cathérine Dorothée PEETERS, Hertogenbosch (NL)

(73) Assignee: NEUROPHYXIA B.V., 's-Hertogenbosch (NL)

(21) Appl. No.: 18/551,793

(22) PCT Filed: Mar. 23, 2022
(86) PCT No.: PCT/NL2022/050153

§ 371 Date: Sep. 21, 2023

(30) Foreign Application Priority Data
Mar. 23, 2021 (NL) 2027799

(51) Int. Cl. A61K 31/4188; A61K 38/49

(52) U.S. Cl. CPC A61K 31/4188; A61K 38/49; C12Y 304/21068

(57) ABSTRACT

The invention relates to the field of medicine. In particular, the present invention relates to the use of 2-iminobiotin for use in the treatment of stroke, wherein 2-iminobiotin is administered in combination with a fibrinolytic drug. The invention in particular relates to the use of 2-iminobiotin for use in the treatment of acute ischemic stroke is due to vessel occlusion eligible for endovascular mechanical thrombectomy.

The invention relates to the field of medicine. In particular, the present invention relates to the use of 2-iminobiotin (2-IB) for use in the treatment of ischemic stroke, wherein 2-IB is administered in combination with a fibrinolytic drug.

### BACKGROUND

Stroke is the second leading cause of death, with an incidence of about 16 million worldwide. The prevalence of stroke among the US population increases with age starting with 2.7% among people 20 years of age, 6% over 60 years and reaching 13% for people above 80 years. Each year, there are ˜800,000 new or recurrent cases of stroke. Patients who survive stroke may end up with long-term disability, requiring rehabilitation with an associated annual cost of more than US$40 billion in the USA alone. In addition, the disability burden attributed to stroke continues to grow, with an estimated increase to 68 million disability-adjusted life years in 2020 [1, 2].

The importance of early reperfusion for improved clinical outcomes has been established by several studies [3, 4, 5, 6]. Every 30 min delay in mechanical thrombectomy decreases favorable outcomes by 11% [7] and every 15 min acceleration at initiating tissue plasminogen activator (t-PA) is associated with 4% greater odds of walking independently at discharge [8]. Throughout the world, individual institutions and the healthcare system overall have in the past decades tried to reduce the time for stroke treatment [9, 10, 11].

Stroke is the second most common cause of mortality (responsible for >6 million deaths annually) and the second most common cause of disability. The absolute number of people with stroke and the global burden of stroke-related disability is high and increasing [2, 12, 13]. The associated annual direct and indirect costs are estimated at more than 40 billion dollars in the US alone [2].

The estimated global lifetime risk of stroke for adult men and women is about 25 percent [12].

In Europe the annual stroke incidence rates are approximately 100 per 100.000 with considerable differences between countries [14]. In the Netherlands more than 25.000 acute ischemic stroke (AIS) patients are admitted to hospitals annually [15].

Large vessel occlusions (LVOs), i.e., arterial blockage in the proximal intracranial circulation, represent an important subpopulation of AIS with an estimated annual incidence of 24 per 100.000 people per year [16]. AIS due to LVO account for about 30% of all AIS, but are disproportionately responsible for 60% of all stroke-related dependency and 90% of mortality [16, 17]. The effect of intravenous thrombolysis (IVT) with Alteplase is limited for AIS caused by an LVOs in the anterior intracranial circulation. Best medical management, including intravenous thrombolysis (IVT) resulted in poor clinical outcome (death or dependency at follow up) in >80% of LVO patients in the control group of the pivotal MR CLEAN trial [18].

Since 2015, several trials have demonstrated that modern endovascular therapy (EVT) can effectively recanalize blocked major arteries in patients with AIS due to LVO with high reperfusion rates (>70% successful reperfusion) [19]. Although catheter based intra-arterial EVT after IVT improves clinical outcomes for 20% of AIS patients caused by LVO, more than half of AIS patients due to LVO still do not regain an independent lifestyle [19]. Moreover, only 10% of LVO patients are symptom-free at follow-up despite combined IVT and EVT treatments [19]. This leaves a great unmet need for AIS patients due to LVO and an opportunity for additional new stroke therapies.

In the last decade knowledge of the molecular and cellular mechanisms impaired by cerebral ischemia has increased, highlighting the role of excitotoxicity, inflammation, oxidative stress, and depolarization waves as pathways and targets to promote neuroprotection avoiding neuronal damage [20, 21]. Cytoprotection aims to augment the resilience of neurons, neurovascular units, and white matter during ischemia until perfusion is restored. Several cytoprotection strategies have been reported to reduce brain infarction in preclinical models of ischemic stroke but there are currently no evidence-based pharmacological neuroprotectants in humans [22, 23].

Preclinical studies have demonstrated that neuroprotection is of greatest benefit for improving functional outcome in experimental animal stroke models in which stroke is followed by reperfusion, as compared with stroke models with permanent arterial occlusion [24]. The recent advent of EVT provides an opportunity to reassess neuroprotection as an adjunctive therapy in patients with types of temporary brain ischemia that align more closely with successful preclinical models [24]. Recently, the ESCAPE NA1 investigators showed in a multicenter EVT trial, that a single dose of the neuroprotectant nerinetide produced a significant improvement among stroke patients that did not receive alteplase, whereas nerinetide did not show favorable outcomes in alteplase-treated stroke patients, likely due to drug-drug interaction with reduced nerinetide concentrations in the Alteplase group [24].

In randomized trials of acute ischemic stroke, intravenous thrombolysis (IVT) with alteplase reduced the risk of a poor outcome [49, 50]. However, two thirds of the patients treated with IVT within 3 hours of stroke onset in these trials were dead or dependent at the end of follow-up. In MR CLEAN, a landmark randomized clinical trial (RCT), 67% of the patients in the endovascular treatment arm were dead or dependent at three months. The high risk of a poor outcome, even after these acute revascularization strategies, may to a large extent be explained by no-reflow. No-reflow has been linked to distal micro vascular damage or dysfunction as a result of tissue necrosis and cell death, or the intervention simply being late. Currently the role of IVT in acute ischemic stroke treatment with IAT is unclear [51].

For large, proximally located thrombi the lytic effect of intravenous alteplase is limited. Given the potential risk of hemorrhage, limited and the cost of intravenous alteplase, investigators have questioned the need for intravenous thrombolysis if patients are eligible to undergo endovascular thrombectomy and the procedure can be performed in a timely fashion.

In patients with acute ischemic stroke due to large-vessel occlusion in the anterior circulation who were eligible for treatment with both intravenous alteplase and endovascular thrombectomy according to most guidelines, direct intervention with intraarterial thrombectomy was noninferior to the combination of initial intravenous alteplase followed by endovascular thrombectomy [52].

In addition to the limited time window and contraindications to IVT, the suboptimal effect observed in today's treatment, including but not limited to the adverse effects seen with the use of tissue plasminogen activators, like Alteplase, necessitates development of alternative method of treatments of stroke, addressing at least one of the drawbacks of the current treatment methods.

### SUMMARY OF THE INVENTION

In a first embodiment, the invention provides 2-iminobiotin (2-IB) in combination with a fibrinolytic drug, in particular Alteplase, for use in the treatment of an individual suffering from AIS.

In certain embodiments, 2-IB for use according to the invention is provided, wherein the AIS is due to vessel occlusion eligible for endovascular mechanical thrombectomy, preferably due to large, medium or distal vessel occlusion.

In some embodiments, 2-IB for use according to the invention is provided, wherein the individual undergoes, is scheduled to undergo, or has undergone EVT. In some embodiments, the AIS is followed by reperfusion of ischemic brain tissue. In some embodiments, 2-IB selectively inhibits neuronal nitric oxide synthase (nNOS), selectively inhibits inducible nitric oxide synthase (iNOS), or selectively inhibits nNOS and iNOS.

In some embodiments, administration of 2-IB to the individual is started within 24 hours after stroke onset or last seen well, preferably within 18 hours, more preferably within 12 hours, most preferably within 8 hours after stroke onset or last seen well and/or administration of 2-IB to the individual is started within 6 hours after the diagnosis AIS due to LVO.

In some embodiments, administration of 2-IB to the individual is started within 2 hours, preferably within 1 hour, preferably within 15 minutes after the individual has undergone EVT and EVT is completed, preferably with successful reperfusion in the angiosuite.

In some embodiments, 2-IB is administered as a loading dose, and/or a continuous infusion for a duration of between 4-6 hours or for a duration of between 6-48 hours. The loading dose may be between 1.5-5 mg, or between 2-3 mg, or between 2-2.5 mg, or about 2.25 mg. In some embodiments, 2-IB is administered as a continuous infusion of between 0.25-3 mg/hour, or between 0.5-2.25 mg/hour, or between 0.75-1.5 mg/hour, or about 1 mg/hour In some embodiments, the 2-IB dose administered as a continuous infusion is adjusted based on the individual's estimated glomerular filtration rate (eGFR). In some embodiments, 2-IB is administered in a loading dose, followed by a continuous infusion, wherein in at least the last 12 hours, preferably at least the last 16 hours, more preferably at least the last 20 hours of the continuous infusion, the 2-IB dose is adjusted based on the individual's eGFR.

2-IB for use according to the invention may, inter alia, result in lower NIH stroke scale (NIHSS) at 24 hours, at about 5-7 days after treatment and/or at about 90 days after treatment. It may also result in lower scores on the modified Rankin Scale (mRS) for the evaluation of neurological functional disability when assessed at about 90 days, or in lower mortality rates when assessed at hospital discharge, at 7 days, and/or about 90 days after AIS.

Other outcomes of 2-IB for use according to invention may be: smaller infarct volume when measured with MRI at 24-48 hours (or CT in case of contraindication for MRI), less embolization in new territory on angiography during EVT or infarction in new territory on 24-48 h computed tomographic angiography (CTA), magnetic resonance angiography (MRA), or intra-arterial digital subtraction angiography (DSA), lower stroke severity, less severe depressive disorder in the individual, improved quality of life of the individual, improved cognitive function of the individual, lower neurofilaments levels in blood of the individual at 24-72 hours, at hospital discharge, or after 3 months after AIS, or lowered risk of postintervention intracranial hemorrhage on neuroimaging according to the Heidelberg Bleeding Classification within 24 hours of study drug administration.

In certain embodiments, 2-IB for use according to the invention is provided, wherein 0.1 to 10 mg/kg/day of 2-IB, or 0.2 to 5 mg/kg/day of 2-IB, or 0.2 to 1 mg/kg/day of 2-IB is administered to the individual. Some embodiments provide 2-IB for use according to the invention, wherein 2-IB is administered such that the area under the plasma concentration time curve from 0 to 4 h is between 100 ng·h/mL to 2000 ng·h/mL. In some embodiments, 2-IB is administered intravenously. In some embodiments, 2-IB is administered intraarterially.

### DETAILED DESCRIPTION OF THE INVENTION

The invention thus provides 2-IB for use in the treatment of an individual suffering from AIS, wherein 2-IB is administered in combination with a fibrinolytic drug.

In some embodiments, the fibrinolytic drug is a t-PA, such as Alteplase. Alteplase is a manufactured form of tissue plasminogen activator, was approved for medical use in the United States in 1987, and is on the World Health Organization's List of Essential Medicines. Alteplase works by converting plasminogen to plasmin in a blood clot. Common side effects are bleeding including intracranial bleeding and gastrointestinal bleeding. Alteplase is contraindicated in patients with recent surgery or trauma, bleeding within the brain, uncontrolled high blood pressure, clotting defects, aneurysm or tumor in the brain and active bleeding. Other examples of t-PAs are Reteplase, a genetically engineered, smaller derivative of recombinant t-PA and Tenecteplase, which has a longer half-life and greater binding affinity for fibrin than recombinant t-PA. Currently, Alteplase is the only t-PA which is approved by the FDA for cerebrovascular thrombotic stroke treatment.

Because of the adverse effects of t-PA treatment, in particular intracranial and gastrointestinal bleeding, the fact that the use of 2-IB and t-PA work synergistically in individuals suffering from acute stroke leads to the possibility of lowering the t-PA dose, thereby improving outcome of patients treated with 2-IB. In some embodiments, 2-IB and the t-PA, preferably Alteplase, are administered separately. In some embodiments, 2-IB and the t-PA, preferably Alteplase are administered simultaneously. In this context, simultaneously means: at the same time, e.g., in two separate lines in an infusion or, together, in one infusion. Separately in this context means: not at the same time, e.g., 2-IB is administered as a bolus, followed by an infusion of the t-PA, or vice versa. A combination of separate and simultaneous infusion is also possible, wherein first one of the two is administered, followed by the other and, thereafter, both drugs are administered simultaneously in one or multiple infusions. In a preferred embodiment, the tPA dose is half of the recommended dosing without concomitant exposure to 2-IB. For Alteplase, for instance, the recommended mono-therapy dose for an individual is 0.9 mg/kg IV with a maximum of 90 mg total. In combination with 2-IB the preferred dose is 0.45 mg/kg with a maximum of 45 mg. More preferred is even a lower dose, e.g., a third, or even a sixth of the recommended mono-therapy dose.

2-IB has been proven to be safe in healthy volunteers as well as in preclinical safety and Phase I and II clinical studies in neonates with asphyxia and adults after cardiac arrest, hence no safety concerns with regard to the use of 2-IB according to the invention are to be expected [28, 29, 30]. In a preferred embodiment, 2-IB for use according to the invention is provided, wherein the AIS is due to vessel occlusion eligible for EVT, preferably due to large, medium or distal vessel occlusion. In particular wherein the AIS is due to symptomatic intracranial vessel occlusion, preferably an occlusion of the internal carotid artery (ICA), one of the first and second segments of the middle cerebral artery (MCA M1 or M2), the anterior cerebral artery, the vertebral artery, the basilar artery, or the proximal posterior cerebral artery, more preferably an occlusion in the anterior circulation, most preferably an occlusion of the internal carotid artery (ICA) or one of the first and second segments of the middle cerebral artery (MCA M1 or M2).

Two key distinctive anatomic features of distal, medium cerebral arteries profoundly affect endovascular procedure conduct and endovascular device design: (1) vessel distance/tortuosity and (2) vessel size [31]. The intermediate, “medium vessels” can be operationally defined as cerebral arteries with lumen diameters between 0.75 and 2.0 mm. The upper 2.0-mm threshold places into the large vessel category the intracranial internal carotid artery (ICA; typical diameter, 3.8 mm), the M1 segment of the MCA (2.7 mm), the basilar artery (3.2 mm), and the vertebral artery (2.8 mm) [32, 33, 34]. There is wide agreement in the literature that the proximal, large artery category includes the intracranial ICA, M1 MCA segment, intracranial vertebral arteries, and basilar artery. Similarly, a general consensus recognizes the distal, middle artery category as including the M3 and M4 MCA segments, A2 to A5 ACA segments, P2 to P5 PCA segments, and PICA, AICA, and SCAs. However, categorization of M2 MCA, A1 ACA, and P1 PCA has varied. Positioning of the M2 MCA within any classification system is particularly challenging, as M2 MCA angioarchitecture is highly heterogenous across patients [35]. Within the current invention, therefore, it preferred to refer to the different arteries by name and not by category.

It is preferred to provide 2-IB for use according to the invention, wherein the individual undergoes, is scheduled to undergo, or has undergone EVT, more preferably within 24 hours after stroke onset or last seen well. 2-IB for use as provided by the invention is in particular useful if the obstruction that diminishes blood flow to the brain is removed and 2-IB is given early after stroke onset. One option to resolve the obstructive blood cloth is the use of fibrinolytic medication, such as Alteplase, but in particular for large obstructions (in the large or medium vessels), the use of Alteplase may not the best possible treatment option. In such cases, EVT is the most useful treatment option to remove the obstruction.

In a preferred embodiment, the present invention provides 2-IB for use according to any one of the preceding claims, wherein the AIS is followed by reperfusion of ischemic brain tissue. Previously it has been demonstrated that 2-IB prevents (further) tissue damage after ischemia-reperfusion, in all likelihood by selectively inhibiting inducible and/or neuronal nitric oxide synthase. In one preferred embodiment, therefore, 2-IB for use according to the invention is provided, wherein 2-IB selectively inhibits nNOS, selectively inhibits inducible iNOS, or selectively inhibits nNOS and iNOS. With “selectively inhibits” in this context is preferably meant that nNOS and/or iNOS are inhibited relatively more than endothelial nitric oxide synthase (eNOS). More preferably, nNOS and/or iNOS are inhibited at least more than 20%, more preferably at least more than 40%, more preferably at least 60% relative to eNOS.

The sooner 2-IB is administered after an ischemic insult, the better the protective effect on reperfusion damage. It is thus preferred that administration of 2-IB to the individual is started within 24 hours after stroke onset or last seen well, preferably within 18 hours, more preferably within 12 hours, more preferably within 8 hours, most preferably within 6 hours after stroke onset or last seen well. It is further preferred that administration of 2-IB to the individual is started within 6 hours after the diagnosis AIS due to LVO, preferably after CTA, MRA, or DSA [36].

In one preferred embodiment, 2-IB for use according to the invention is provided, wherein administration of 2-IB to the individual is started within 2 hours, preferably within 1 hour, preferably within 15 minutes after the individual has undergone EVT and EVT is completed, preferably with successful reperfusion in the angiosuite, said successful reperfusion preferably exceeding expanded Thrombolysis in Cerebral Infarction (eTICI) grade 2b50, more preferably exceeding grade 2b67, more preferably exceeding grade 2c, most preferably equaling grade 3. Revascularization after EVT for AIS is preferably measured by the eTICI scale. In brief, eTICI grade 0 is equivalent to no reperfusion or 0% filling of the downstream territory; eTICI 1 reflects thrombus reduction without any reperfusion of distal arteries; eTICI 2a is reperfusion in less than half or 1-49% of the territory; eTICI 2b50 is 50-66% reperfusion; eTICI 2b67 is 67-89% reperfusion, eTICI 2c is equivalent to 90-99% reperfusion; and eTICI 3 is complete or 100% reperfusion [37].

It is preferred that 2-IB is administered intra-arterially. In some embodiments, it is preferred that 2-IB is administered intra-venously. It is further preferred that 2-IB is administered as a bolus or loading dose, and/or a continuous infusion for a duration of between 4-6 hours or for a duration of between 6-48 hours, preferably 12-36 hours, more preferably 20-28 hours, most preferably for about 24 hours. In some embodiments, 2-IB is preferably administered in a loading dose of between 1.5-5 mg, preferably of between 2-3 mg, more preferably of between 2-2.5 mg, most preferably of about 2.25 mg. In some embodiments, 2-IB is preferably administered as a continuous infusion of between 0.25-3 mg/hour, preferably of between 0.5-2.25 mg/hour, preferably of between 0.75-1.5 mg/hour, most preferably of about 1 mg/hour. In some embodiments, it is preferred that during at least part of the continuous infusion, the 2-IB dose is adjusted based on the individual's estimated glomerular filtration rate (eGFR). eGFR is based on serum creatinine levels and takes into account both the age and sex of the individual. An exemplary formula for determining eGFR is as follows:

eGFR=186×[Creatinine(μmol/l)/88.4]^{-1.154}×[Age (years)]^{-0.203}×[0.724 if female]×[1.21 if black].

This is one formula for determining eGFR. Other formulas with similar outcome are described, e.g., in Rule et al [38].

eGFR adjusted doses are particularly useful for individuals having a high GFR. Accordingly, the disclosure provides methods for determining a dosage of 2-IB for treating brain cell injury in an individual, the method comprising determining the eGFR in the individual and adjusting the 2-IB dosage based on the eGFR. An exemplary dosing scheme based on eGFR is as follows, however a skilled person will recognize that the dosages may vary as much as 10 or 20% (+/−) as listed below.

TABLE 1
Exemplary dosing scheme of a 2-IB formulation having 0.75 mg 2-IB
per ml, when corrected for eGFR of a patient
(Total volume = total volume of infusion per 20 hours). If, for instance
because the patient was hospitalized, the eGFR is already known before
starting 2-IB administration, the eGFR adjusted dose can be
administered for the full 24 hours and the total volume
in the table below is adjusted accordingly.

| eGFR | Total volume | Pump speed |
|---|---|---|
| (ml/min/1.73 m²) | ml | (ml/u) |
| 20-29 | 9 | 0.5 |
| 30-39 | 12 | 0.6 |
| 40-49 | 15 | 0.8 |
| 50-59 | 18 | 1.0 |
| 60-69 | 22 | 1.1 |
| 70-79 | 26 | 1.3 |
| 80-99 | 30 | 1.5 |
| 100-124 | 40 | 2.0 |
| 125-149 | 48 | 2.4 |
| 150-174 | 55 | 2.8 |
| 175-199 | 65 | 3.3 |
| 200-220 | 70 | 3.5 |

It is preferred that at least part of the continuous infusion is adjusted based on the individual's EGFR but not the loading dose. This is due to the fact that administration of 2-IB has to start as soon as possible, which means that there is no time to wait for the eGFR determination prior to administering the loading dose and/or the first part of the continuous infusion. Therefore, in a preferred embodiment, 2-IB for use according to the invention is provided, wherein 2-IB is administered in a loading dose, followed by a continuous infusion, wherein in at least the last 12 hours, preferably at least the last 16 hours, more preferably at least the last 20 hours of the continuous infusion, the 2-IB dose is adjusted based on the individual's eGFR. In some instances, in particular when a second stroke occurs during treatment with 2-IB, the continuous infusion is preferably prolonged. In such case, the infusion is preferably continued for another at least 12 hours, preferably at least 16 hours, more preferably at least 20 hours from the timepoint of the second stroke.

In some embodiments, 2-IB for use according to the invention is provided, wherein 0.1 to 10 mg/kg/day of 2-IB, preferably 0.2 to 5 mg/kg/day of 2-IB more preferably 0.2 to 1 mg/kg/day of 2-IB is administered to the individual. In particular when adjusted based on the individual's eGFR, it is preferred to administer 2-IB such that the area under the plasma concentration time curve from 0 to 4 h is between 100 ng·h/mL to 2000 ng·h/mL, more preferably between 300 ng·h/ml to 1300 ng·h/ml, most preferably between 365-1018 ng·h/mL. In the field of pharmacokinetics, the area under the curve (AUC) is the definite integral of a curve that describes the variation of a drug concentration in blood plasma as a function of time, and represents the total drug exposure across time. In practice, the drug concentration is measured at certain discrete points in time and the AUC is estimated using the trapezoidal rule, a technique for approximating the definite integral. One use of the AUC is in the therapeutic drug monitoring of drugs with a narrow therapeutic index. Previously, it was observed that within a certain range around 30 ng/ml in a neuronal culture, 2-IB is most active in vitro [39]. The effect of 2-IB can be measured in several ways and at several time points after treatment. It is preferred that treatment of 2-IB leads to an improvement of at least one of the many symptoms associated with stroke, such improvement, e.g., being: a lowering of the NIHSS, a lower score on the modified Rankin scale, lower mortality, smaller infarct volume, less embolization, less severe depression, improved quality of life, improved cognitive function, lower neurofilaments levels in blood, or lower risk of postintervention intracranial hemorrhage. In the following section a more detailed description of these symptoms and methods for measuring them is given. Improvement in this context means that the respective parameter is improved, i.e., higher or lower depending on the parameter, in a patient group that received 2-IB treatment according to the invention in comparison with a group of patients that did not receive 2-IB. The group of patients that did not receive 2-IB may also be a suitably selected historical cohort. Such suitable historical cohort can be easily identified, based on the demographic values of the group of patients that is treated with 2-IB.

In one embodiment, 2-IB for use according to the invention is provided, wherein the treatment results in lower stroke severity, when measured using the NIHSS at 24 hours, at about 5-7 days after treatment and/or at about 90 days after treatment. The NIHSS is a 15-item impairment scale used to measure stroke severity. It was originally developed in 1989 and is now a widely used outcome measure in the recombinant t-PA stroke trials. In the current National Stroke Foundation guidelines, the NIHSS is recommended as a valid tool to assess stroke severity in emergency departments and captures both motor and non-motor impairments of stroke, and provides a good overview of people's deficits [40]. A lower NIHSS in this respect means a lower NIHSS in a group of patients that were treated with 2-IB in comparison with a group of patients that did not receive 2-IB. In one embodiment, the group of patients that did not receive 2-IB is a suitably selected historical cohort.

In one embodiment, 2-IB for use according to the invention is provided, wherein the treatment results in a lower score on the modified Rankin Scale (mRS) for the evaluation of neurological functional disability when assessed at about 90 days. The mRS comprises seven “grades” of handicap: 0 No symptoms; 1 Minor symptoms that do not interfere with lifestyle; 2 Minor handicap, symptoms that lead to some restriction in lifestyle but do not interfere with the patient's capacity to look after himself; 3 Moderate handicap, symptoms that significantly restrict lifestyle and prevent totally independent existence; 4 Moderately severe handicap, symptoms that clearly prevent independent existence though not needing constant attention; 5 Severe handicap, totally dependent patient requiring constant attention night and day; and 6 is dead [41]. In one embodiment, a lower score on the mRS in this respect means that the mRS is lower in a group of patients that were treated with 2-IB in comparison with a group of patients that did not receive 2-IB. In one embodiment, the group of patients that did not receive 2-IB is a suitably selected historical cohort. One such cohort and the mRS distribution in (stratified) AIS patients could, e.g., be taken from the MR CLEAN Registry [42]. In one embodiment, 2-IB for use according to the invention is provided, wherein the treatment results in lower mortality rates when assessed at about 90 days. Mortality rate is a measure of the number of deaths (in general, or due to a specific cause) in a particular population, scaled to the size of that population, per unit of time. Mortality rate is typically expressed in units of deaths per 1,000 individuals per year; thus, a mortality rate of 9.5 (out of 1,000) in a population of 1,000 would mean 9.5 deaths per year in that entire population, or 0.95% out of the total. In one embodiment, lower mortality rates in this respect means less deaths (in general or due to stroke and/or complications of stroke) in a group of patients that were treated with 2-IB as compared with a group of patients that did not receive 2-IB. In a preferred embodiment, the mortality rate is calculated based on the number of deaths in general. In another preferred embodiment, the mortality rate is calculated based on the number of deaths due to stroke and/or complications of stroke. In one embodiment, the group of patients that did not receive 2-IB is a suitably selected historical cohort. Ois et al, e.g., describes the 90 day mortality in patients with AIS to be about 15.7% [43], whereas Lin et al describes an overall mortality after AIS of 18.7%, which may be lowered to 15.0% by performing EVT [44]. However, it should be considered that the mortality rate in LVO patients is much higher (up to 21%) than in stroke in general [18].

In one embodiment, 2-IB for use according to the invention is provided, wherein the treatment results in smaller infarct volume when measured with MRI at 24-48 hours (or CT in case of contraindication for MRI). A skilled person is aware how to calculate infarct volume based on MRI or CT scan results, e.g., as described in Kim et al or, e.g., with suitable software as RAPID AI (rapidai.com) or any other validated software program. In one embodiment, a smaller infarct volume in this respect means that the infarct volume is smaller in a group of patients that were treated with 2-IB in comparison with a group of patients that did not receive 2-IB. In one embodiment, the group of patients that did not receive 2-IB is a suitably selected historical cohort. Kim et al (2019), e.g., describe a infarct volume (percent of brain volume) of between 0.29-4.18% (mean: 0.93%) in their patient population. As said, however, the skilled person is able to select a suitable patient cohort based on the demographic analysis of the patient group that received 2-IB in order to compare said treated group with the (historical) untreated group.

In one embodiment, 2-IB for use according to the invention is provided, wherein the treatment results in less embolization in a new territory on angiography during EVT or infarction in new territory on 24-48 h CTA, MRA of DSA.

In one embodiment, 2-IB for use according to the invention is provided, wherein treatment results in less severe depressive disorder in the individual, when measured with structured interviews, such as the Structured Clinical Interview for Depression (SCID), or the Depression Interview and Structured Hamilton (DISH), assessed at 30-90 days. A less severe depressive disorder in this respect means less severe in a group of patients that were treated with 2-IB in comparison with a group of patients that did not receive 2-IB. In one embodiment, the group of patients that did not receive 2-IB is a suitably selected historical cohort.

In one embodiment, 2-IB for use according to the invention is provided, wherein treatment results in improved quality of life of the individual, when measured with the EQ-5D questionnaire and/or the hospital anxiety and depression scale (HADS). Both tests return a lower score when quality of life is improved. A lower score in this respect means a lower score in a group of patients that were treated with 2-IB in comparison with a group of patients that did not receive 2-IB. In one embodiment, the group of patients that did not receive 2-IB is a suitably selected historical cohort.

In one embodiment, 2-IB for use according to the invention is provided, wherein treatment results in improved cognitive function of the individual, when measured with higher scores in the Montreal Cognitive Assessment (MoCA) [46]. A higher score in this respect means a preserved cognition in a group a patients that were treated with 2-IB in comparison with a group of patients that did not receive 2-IB. In one embodiment, the group of patients that did not receive 2-IB is a suitably selected historical cohort.

In one embodiment, 2-IB for use according to the invention is provided, wherein treatment results in lower neurofilaments levels in blood of the individual at 24-72 hours. In one embodiment, 2-IB for use according to the invention is provided, wherein treatment results in lower neurofilaments levels in blood of the individual at 1 month, 2 months, or 3 months. Neurofilament light chain (NfL) is a promising biomarker for predicting tissue damage or stroke recurrence [47]. A lower level in this respect means a lower level in a group of patients that were treated with 2-IB in comparison with a group of patients that did not receive 2-IB. In one embodiment, the group of patients that did not receive 2-IB is a suitably selected historical cohort.

In one embodiment, 2-IB for use according to the invention is provided, wherein the treatment results in a lower risk of postintervention intracranial hemorrhage on neuroimaging according to the Heidelberg Bleeding Classification within 24 hours of study drug administration. A lower risk in this respect means a lower risk in a group of patients that were treated with 2-IB in comparison with a group of patients that did not receive 2-IB. In one embodiment, the group of patients that did not receive 2-IB is a suitably selected historical cohort.

### EXAMPLES

### Example 1—Phase II Study

### Objectives, Study Design:

The study is a prospective, Phase 2, open label, single centre study with as primary objective the evaluation of safety and tolerability of 2-IB, and as secondary objective the gathering of preliminary data on efficacy. Patients are included in 4 groups. All patients will receive 2-IB in a fixed loading dose of 3 mL, followed by continuous intravenous administration for a duration of maximally 24 hours.

TABLE 1
overview of the 4 groups

| | Start 2-IB directly at | |
|---|---|---|
| | diagnosis | Start 2-IB at successful reper- |
| | of AIS due to LVO | fusion after EVT |
| IVT followed by | Group 1a | Group 2a |
| EVT | | |
| Only EVT | Group 1b | Group 2b |

Group 1a + 1b: n = 9
Group 2a + 2b = 9

A further group is formed from patients treated before the current study (historical cohort) that were not treated with 2-IB. This group is established from patients treated in the same hospital, and is based on the demographics of the current study population.

A 2-IB dose based on the eGFR on the basis of pharmacokinetics and safety of 2-IB in a phase II, open-label, dose-escalation intervention study in adult human patients after cardiac arrest is used, in which this eGFR adjusted dose targeted the AUC more precisely and was tolerated without clinical drug-related adverse events.

### Main Study Parameters/Endpoints:

The main study parameters used for evaluating the short-term safety and tolerability are vital signs (heart frequency, blood pressure and oxygen saturation) before and during 24 hours after administration of the study drug and the need for clinical intervention. Furthermore, the occurrence of (Serious) Adverse Events ((S)AEs) until 7 days on the neurology department or until discharge from the neurology department, whichever occurs earlier. For evaluation of the pharmacokinetics profile of 2-IB, 4 plasma samples are analyzed at different intervals (4 hours, 24 hours, 25 hours, and 28 hours after study medication). Pharmacokinetic parameters include Cmax, AUC, Tmax, T1/2, clearance (CI), and volume of distribution (Vd).

### Secondary Parameters:

1. Stroke Severity measured with the NIHSS
2. Long term safety as determined by the occurrence of SAEs
3. Score on the modified Rankin Scale (mRS) at 90 days
4. Score on the HADS (hospital anxiety and depression scale) and EQ-5D questionnaire
5. Successful vessel recanalization defined as extended Thrombolysis in Cerebral Infarction (eTICI) grade 2b, 2c or 3 on the post-procedure angiogram
6. Infarct volume measured with MRI at 24-48 hours
7. Embolization in new territory on angiography during EVT or infarction in new territory on 24-48 h MRI (or CT)
8. Vessel recanalization at 24 hours MRA (or CTA in case of contraindications for MRI)
9. Any postintervention intracranial hemorrhage on neuroimaging within 24 hours.
10. Blood investigation (CBC, electrolytes, serum creatinine, serum glucose) at 24 hours and a single biomarker neurofilament light chain at 24 hours.

SCHEDULE OF ASSESSMENTS

| | | | | | 7 days | | |
|---|---|---|---|---|---|---|---|
| | | | | 24 hours | after stroke | 30 days | |
| | Before the | After the | During | after stroke | onset or | after stroke | |
| | first dose | first loading | first | onset | discharge | onset | 90 days |
| | of 2-IB | dose | 24 hours | (±12 hours) | (±2 days) | (±7 days) | (±14 days) |
| Selection criteria | X | | | | | | |
| History, prior medication | X | | | | | | |
| and examination | | | | | | | |
| including weight | | | | | | | |
| NIHSS score | X | | | X | X | | |
| Vital signs | X | X | X* | | | | |
| CBC, electrolytes, serum | X | | | X | | | |
| creatinine, serum glucose | | | | | | | |
| Plasma PK sampling** | | | X** | X** | | | |
| Neurological biomarker | | | | X*** | | | X*** |
| ECG | X | | X**** | | | | |
| Neuroimaging | X | | | X | | | |
| Recording of AEs | | X | | X | X | X***** | |
| Concomitant medications | | | | | X | | X |
| Drug accountability | | | | X | | | |
| Informed consent | | | | X****** | | | |
| modified Rankin Scale | X | | | | | | X |
| (including mortality) | | | | | | | |
| HADS (hospital anxiety | | | | | | | X |
| and depression scale) | | | | | | | |
| EQ-5D | | | | | | | X |

*Vital signs: heart rate, blood pressure, temperature and oxygen saturation will be documented at the emergency department in the hospital information system, vital signs are continuously monitored in the angiosuite during EVT and subsequently vital signs will be documented in the hospital information system at the neurocare unit in the following manner: 0 to 2 hours after admission each 15 minutes, 2 to 8 hours after admission each 30 minutes, 8 to 24 hours after admission each 60 minutes, temperature will be documented 0-24 hours each 6 hours
**PK samples (3.5 mL) will be withdrawn after 4 hours, 24 hours, 25 hours and 28 hours after start of the first administration (with a margin of ±15 min)
***Blood sample (3.5 mL) for neurological biomarker neurofilament light chain will be withdrawn at 24 h (with a margin of ±2 hours) and at 90 days (with a margin of 10 days)
****ECG will repeated only if relevant changes are noted during continuous monitoring (first 24 hours at the neurocare unit or intensive care unit)
*****From Day 7 (or from discharge from the neurology ward, whichever is earlier) until Day 30 after AIS onset, only SAE's will be recorded.
******As soon as possible, within 72 hours after admission

### Study Population

The study population consists of adults (age ≥18 years for males and age >49 for female patients) with AIS due to LVO. This study consists of 2 subgroups: patients treated with IVT (Alteplase), followed by EVT (n=12); and patients only treated with EVT (n=6). Subjects receive 2-IB on top of standard treatment.

The study population is drawn from patients with a clinical diagnosis of AIS at the Emergency Department of the HMC. Patients meeting the inclusion and exclusion criteria as set below are entered in the study.

### Inclusion Criteria

Adults (age >18 years for males and age >49 years for females)
A clinical diagnosis of AIS
Baseline NIH stroke scale score ≥4
Alberta Stroke Program Early CT score (ASPECTS)>4 on CT (or MRI)
Presence of an intracranial LVO of the anterior circulation (distal ICA, M1 or M2 segment of the MCA) on CTA, MRA or DSA
Start of EVT (arterial access puncture) possible within the first 6 hours after stroke onset or last seen well
EVT with declared first endovascular approach as either stent retriever, aspiration device or a combined approach
Written informed consent (after deferred consent)
Pre-stroke independent functional status in activities of daily living (mRS>2)

Note that pretreatment with IV thrombolysis (Alteplase, Tenecteplase or other) is not a prerequisite. Note that CT perfusion results are not used as inclusion criteria.

### Exclusion Criteria

No informed consent
Contraindication to EVT or EVT >6 hours after symptom onset (or last seen well)
Evidence of a large core of established infarction defined as ASPECTS 0-4.
Evidence of absence/poor collateral circulation on CTA (Tan collateral score of 0 or 1) [48].
Known co-morbidity with a life expectancy of <6 months prior to AIS
Women aged 49 or less or known pregnancy*
Cognitive impairment (documented dementia) known prior to ischemic stroke
Pre-stroke disability which interferes with the assessment of functional outcome at 90 days, i.e. mRS >2
Intent to use any endovascular device other than a stent retriever or clot aspiration device or intra-arterial medications as the initial thrombectomy approach.
History of life threatening allergy (more than rash) to contrast medium
Evidence of acute hemorrhage on CT, MRI
Significant mass effect with midline shift.
Subjects with occlusions in multiple vascular territories (e.g., bilateral anterior circulation, or anterior/posterior circulation)
Severe known renal impairment defined as requiring dialysis (hemo- or peritoneal) or if known a creatinine clearance <20 mL/min. *As 2-IB has not been tested yet for embryonic toxicity and limited pre- and postnatal development studies have been performed, women who can be pregnant must not be included be in this study.

For the assessment of safety and tolerability, vital signs, including heart frequency and blood pressure and oxygen saturation (pulse oximeter) are monitored before and until 15 minutes after 2-IB fixed loading dose IV administration. In addition, vital signs are continuously monitored during the first 24 hours of hospital admission which is also part of standard care. Heart rate and blood pressure are documented at the neurocare unit according to the following schedule: 0 to 2 hours after admission each 15 minutes, 2 to 8 hours after admission each 30 minutes, 8 to 24 hours after admission each 60 minutes, body temperature is documented 0-24 hours each 6 hours. Any change in the vital parameters which leads to an intervention is also be documented.

Routine biochemistry and hematology are done before the start of treatment and after the last dose according to clinical protocol.

For the assessment of pharmacokinetics, 4 plasma samples are collected after the first IV loading dose, at 4, 24 hours after the start of 2-IB treatment, and at 1 and 4 hours after stop of 2-IB infusion (i.e. 25 and 28 hours after start of 2-IB treatment).

For safety, all subjects are followed for the duration of their stay in the hospital and all adverse events are recorded in this period.

To gather preliminary signs of short-term efficacy the levels of neurofilaments light at 24 h±2 h after first dose of study drug are evaluated.

After the hospitalization period, all subjects enter the follow-up period of 30 days in which all SAEs are recorded. Neurological functional disability (assessed with the modified Rankin Scale (mRS), including mortality) as well as quality of life as assessed with the EQ-5D questionnaire and the hospital acquired depression scale (HADS) are completed at 90 days (±2 weeks) follow-up. When a patient and his partner are unable to visit the hospital, mRS, HADS and EQ-5D evaluation are done during a standardized telephonic interview.

### Treatment of Subjects

### Investigational Product/Treatment

All patients in the current treatment groups receive open label 2-IB, which is provided in vials of 50 ml. No placebo is used. 2-IB is formulated as a 0.75 mg/ml isotonic, iso-osmotic, saline solution for injection with a low quantity of citric buffer (about 15 mmol/l) at pH 4.0. 2-IB is administered intravenously, preferably through a percutaneously inserted peripheral line. Alternatively, if the patient has a central catheter it is also possible to administer 2-IB through this central line. Study medication is used undiluted and is given as a bolus, followed by a continuous infusion for 24 hours.

Patients receive 2-IB in a fixed loading dose of 3 mL, followed by a 24 hour continuous intravenous infusion. Administration of 2-IB contains the same three parts for all patients.
Part A: Fixed loading dose of 3 mL (concentration 0.75 mg/ml) IV administered in 1 minute
Part B: Standard continuous IV infusion of 1.3 mL per hour for 4 hours from start infusion
Part C: eGFR adjusted continuous IV dose, based on eGFR (as assessed on admission), for the remaining 20 hours. The eGFR will be used for dose titration using the formula: 186×(Creat/88.4)^{-1.154}×(Age)^{-0.203}×(0.742 if female)×(1.210 if black) [22].

### Use of Co-Intervention

Subjects receive other medication and interventions according to normal standard care as determined by the medical doctor in charge. The use of 2-IB will not preclude any concomitant medication or intervention. If an anaphylactoid reaction occurs with either Alteplase or 2-IB, treatment is stopped immediately and appropriate anaphylactoid treatment is given according to local guidelines.

IVT (within 4.5 hours of symptom onset, without contraindications*) is part of usual stroke care. Dose Intravenous Alteplase is 0.9 mg/kg body weight with a maximum of 90 mg, first 10% as bolus, other 90% over 1 hour as an IV infusion.
Contra-indication for treatment with IV Alteplase:
   Arterial blood pressure exceeding 185/110 mmHg and not responding to treatment
   Blood glucose less than 2.7 or over 22.2 mmol/L
   Cerebral infarction in the previous 6 weeks with residual neurological deficit
   Head trauma in the previous 4 weeks
   Major surgery or serious trauma in the previous 2 weeks
   Gastrointestinal or urinary tract hemorrhage in the previous 2 weeks
   Previous intracerebral hemorrhage
   Use of anticoagulant with INR exceeding 1.7 or APTT exceeding 50 seconds
   Known thrombocyte count less than 90×109/L. When the treating physician suspects a thrombocyte count below 90×109/L (e.g. suspected hemorrhagic diathesis), the thrombocyte count in the laboratory should be awaited prior to inclusion.
   Treatment with direct thrombin or factor X inhibitors, unless specific antidote has been given, i.e. idarucizumab in case of dabigatran use.

### Statistical Analysis

Baseline data by treatment allocation will be reported with standard statistical procedures. Missing values for baseline characteristics will be reported. Missing baseline characteristics will be imputed using regression imputation. Additionally, an on-treatment analysis and analysis of safety parameters with all patients, including all patients who had withdrawn from the study, will be performed.

### Primary Study Parameter(s)

Primary safety parameters as described will be recorded per patient and per treatment group and will be listed and summarized in tabular and/or graphical form.

The main study parameters used for evaluating the short-term safety and tolerability will be vital signs (heart frequency, blood pressure and oxygen saturation) before and during 24 hours after administration of the study drug and the need for clinical intervention. Furthermore, the occurrence of (Serious) Adverse Events ((S)AEs) until 7 days on the neurology department or until discharge from the neurology department, whichever occurs earlier.

For evaluation of the pharmacokinetics profile of 2-IB, plasma samples will be analyzed.

Pharmacokinetic parameters to be determined will include Cmax, AUC, Tmax, t1/2, clearance (CI), and volume of distribution (Vd).

### Secondary Study Parameter(s)

Secondary safety and efficacy parameters as described will be recorded per patient and per treatment group and will be listed and summarized in tabular and/or graphical form.

With regards to the efficacy parameters we will assess a preliminary effect by comparison with controls from our LVO stroke registry and/or historical controls.

This preliminary effect estimate will be adjusted for important prognostic factors at baseline, which include age, pre-stroke mRS, time from onset of symptoms to groin puncture, stroke severity (NIHSS), systolic blood pressure, antiplatelet treatment and imaging results at baseline.

### Ethical Considerations

### Regulation Statement

The study will be conducted according to the principles of the Declaration of Helsinki

As stated in the current version of Fortaleza, Brazil, 2013 and in accordance with the Dutch Medical Research Involving Human Subjects Act (WMO).

### REFERENCES

[1] A. Sweid, B. Hammoud, S. Ramesh, D. Wong, T. D. Alexander, J. H. Weinberg, M. Deprince, J. Dougherty, D. J.-M. Maamari, S. Tjoumakaris, H. Zarzour, M. R. Gooch, N. Herial, V. Romo, D. M. Hasan, R. H. Rosenwasser and P. Jabbour, “Acute ischaemic stroke interventions: large vessel occlusion and beyond.,” Stroke and vascular neurology, vol. 5, no. 1, p. 80-85, 2020.
[2] E. J. Benjamin, S. S. Virani, C. W. Callaway, A. M. Chamberlain, A. R. Chang, S. Cheng, S. E. Chiuve, M. Cushman, F. N. Delling, R. Deo, S. D. de Ferranti, J. F. Ferguson, M. Fornage, C. Gillespie, C. R. Isasi, M. C. Jiménez, L. C. Jordan, S. E. Judd, D. Lackland, J. H. Lichtman, L. Lisabeth, S. Liu, C. T. Longenecker, P. L. Lutsey, J. S. Mackey, D. B. Matchar, K. Matsushita, M. E. Mussolino, K. Nasir, M. O'Flaherty, L. P. Palaniappan, A. Pandey, D. K. Pandey, M. J. Reeves, M. D. Ritchey, C. J. Rodriguez, G. A. Roth, W. D. Rosamond, U. K. A. Sampson, G. M. Satou, S. H. Shah, N. L. Spartano, D. L. Tirschwell, C. W. Tsao, J. H. Voeks, J. Z. Willey, J. T. Wilkins, J. H. Wu, H. M. Alger, S. S. Wong, P. Muntner, A. H. A. C. on Epidemiology, P. S. Committee and S. S. Subcommittee, “Heart Disease and Stroke Statistics-2018 Update: A Report From the American Heart Association.,” Circulation, vol. 137, no. 12, p. e67-e492, 3 2018.
[3] C.-H. J. Sun, R. G. Nogueira, B. A. Glenn, K. Connelly, S. Zimmermann, K. Anda, D. Camp, M. R. Frankel, S. R. Belagaje, A. M. Anderson, A. P. Isakov and R. Gupta, ““Picture to puncture”: a novel time metric to enhance outcomes in patients transferred for endovascular reperfusion in acute ischemic stroke.,” Circulation, vol. 127, no. 10, p. 1139-1148, 3 2013.
[4] P. Khatri, S. D. Yeatts, M. Mazighi, J. P. Broderick, D. S. Liebeskind, A. M. Demchuk, P. Amarenco, J. Carrozzella, J. Spilker, L. D. Foster, M. Goyal, M. D. Hill, Y. Y. Palesch, E. C. Jauch, E. C. Haley, A. Vagal, T. A. Tomsick and I. M. S. I. I. I. Trialists, “Time to angiographic reperfusion and clinical outcome after acute ischaemic stroke: an analysis of data from the Interventional Management of Stroke (IMS III) phase 3 trial.,” The Lancet. Neurology, vol. 13, no. 6, p. 567-574, 6 2014.
[5] M. Goyal, A. P. Jadhav, A. Bonafe, H. Diener, V. Mendes Pereira, E. Levy, B. Baxter, T. Jovin, R. Jahan, B. K. Menon, J. L. Saver and S. W. I. F. T. P. R. I. M. E. investigators, “Analysis of Workflow and Time to Treatment and the Effects on Outcome in Endovascular Treatment of Acute Ischemic Stroke: Results from the SWIFT PRIME Randomized Controlled Trial.,” Radiology, vol. 279, no. 3, p. 888-897, 6 2016.
[6] B. K. Menon, T. T. Sajobi, Y. Zhang, J. L. Rempel, A. Shuaib, J. Thornton, D. Williams, D. Roy, A. Y. Poppe, T. G. Jovin and others, “Analysis of workflow and time to treatment on thrombectomy outcome in the endovascular treatment for small core and proximal occlusion ischemic stroke (ESCAPE) randomized, controlled trial,” Circulation, vol. 133, p. 2279-2286, 2016.
[7] Z.-S. Shi, D. S. Liebeskind, B. Xiang, S. G. Ge, L. Feng, G. W. Albers, R. Budzik, T. Devlin, R. Gupta, O. Jansen, T. G. Jovin, M. Killer-Oberpfalzer, H. L. Lutsep, J. Macho, R. G. Nogueira, M. Rymer, W. S. Smith, N. Wahlgren, G. R. Duckwiler, T. R. E. V. O. Multi MERCI and T. R. E. V. O. 2. Investigators, “Predictors of functional dependence despite successful revascularization in large-vessel occlusion strokes.,” Stroke, vol. 45, no. 7, p. 1977-1984, 7 2014.
[8] B. S. Katz, O. Adeoye, H. Sucharew, J. P. Broderick, J. McMullan, P. Khatri, M. Widener, K. S. Alwell, C. J. Moomaw, B. M. Kissela, M. L. Flaherty, D. Woo, S. Ferioli, J. Mackey, S. Martini, F. De Los Rios la Rosa and D. O. Kleindorfer, “Estimated Impact of Emergency Medical Service Triage of Stroke Patients on Comprehensive Stroke Centers: An Urban Population-Based Study.,” Stroke, vol. 48, no. 8, p. 2164-2170, 8 2017.
[9] R. Mikulík, P. Kadlecová, A. Czlonkowska, A. Kobayashi, M. Brozman, V. Svigelj, L. Csiba, K. Fekete, J. Körv, V. Demarin, A. Vilionskis, D. Jatuzis, Y. Krespi, N. Ahmed and S. I. of Treatments in Stroke-East Registry (SITS-EAST) Investigators, “Factors influencing in-hospital delay in treatment with intravenous thrombolysis.,” Stroke, vol. 43, no. 6, p. 1578-1583, 6 2012.
[10] D. Leifer, D. M. Bravata, J. J. Connors III, J. A. Hinchey, E. C. Jauch, S. C. Johnston, R. Latchaw, W. Likosky, C. Ogilvy, A. I. Qureshi and others, “Metrics for measuring quality of care in comprehensive stroke centers: detailed follow-up to Brain Attack Coalition comprehensive stroke center recommendations: a statement for healthcare professionals from the American Heart Association/American Stroke Association,” Stroke, vol. 42, p. 849-877, 2011.
[11] B. P. Mehta, T. M. Leslie-Mazwi, R. V. Chandra, D. L. Bell, C.-H. J. Sun, J. A. Hirsch, J. D. Rabinov, N. S. Rost, L. H. Schwamm, J. N. Goldstein, W. C. Levine, R. Gupta and A. J. Yoo, “Reducing door-to-puncture times for intra-arterial stroke therapy: a pilot quality improvement project.,” Journal of the American Heart Association, vol. 3, no. 6, p. e000963, 11 2014.
[12] V. L. Feigin, E. Nichols, T. Alam, M. S. Bannick, E. Beghi, N. Blake, W. J. Culpepper, E. R. Dorsey, A. Elbaz, R. G. Ellenbogen and others, “Global, regional, and national burden of neurological disorders, 1990-2016: a systematic analysis for the Global Burden of Disease Study 2016,” The Lancet Neurology, vol. 18, p. 459-480, 2019.
[13] V. L. Feigin and G. A. Roth, “Global, regional, and country-specific lifetime risks of stroke, 1990 and 2016,” New England Journal of Medicine, vol. 379, p. 2429-2437, 2018.
[14] P. U. Heuschmann, A. D. Carlo, Y. Bejot, D. Rastenyte, D. Ryglewicz, C. Sarti, M. Torrent and C. D. A. Wolfe, “Incidence of stroke in Europe at the beginning of the 21st century,” Stroke, vol. 40, p. 1557-1563, 2009.
[15] L. S. Kuhrij, M. W. J. M. Wouters, R. M. van den Berg-Vos, F.-E. de Leeuw and P. J. Nederkoorn, “The Dutch acute stroke audit: benchmarking acute stroke care in the Netherlands,” European stroke journal, vol. 3, p. 361-368, 2018.
[16] N. Lakomkin, M. Dhamoon, K. Carroll, I. P. Singh, S. Tuhrim, J. Lee, J. T. Fifi and J. Mocco, “Prevalence of large vessel occlusion in patients presenting with acute ischemic stroke: a 10-year systematic review of the literature,” Journal of neurointerventional surgery, vol. 11, p. 241-245, 2019.
[17] K. Malhotra, J. Gornbein and J. L. Saver, “Ischemic strokes due to large-vessel occlusions contribute disproportionately to stroke-related dependence and death: a review,” Frontiers in neurology, vol. 8, p. 651, 2017.
[18] O. A. Berkhemer, P. S. S. Fransen, D. Beumer, L. A. Van Den Berg, H. F. Lingsma, A. J. Yoo, W. J. Schonewille, J. A. Vos, P. J. Nederkoorn, M. J. H. Wermer and others, “A randomized trial of intraarterial treatment for acute ischemic stroke,” n Engl J Med, vol. 372, p. 11-20, 2015.
[19] M. Goyal, B. K. Menon, W. H. van Zwam, D. W. J. Dippel, P. J. Mitchell, A. M. Demchuk, A. Dávalos, C. B. L. M. Majoie, A. van der Lugt, M. A. De Miquel and others, “Endovascular thrombectomy after large-vessel ischaemic stroke: a meta-analysis of individual patient data from five randomised trials,” The Lancet, vol. 387, p. 1723-1731, 2016.
[20] D. González-Nieto, R. Fernández-Serra, J. Pérez-Rigueiro, F. Panetsos, R. Martinez-Murillo and G. V. Guinea, “Biomaterials to Neuroprotect the Stroke Brain: A Large Opportunity for Narrow Time Windows,” Cells, vol. 9, p. 1074, 2020.
[21] G. J. Hankey, “Nerinetide before reperfusion in acute ischaemic stroke: déjà vu or new insights?,” Lancet (London, England), vol. 395, p. 843, 2020.
[22] H.-S. Sun, T. A. Doucette, Y. Liu, Y. Fang, L. Teves, M. Aarts, C. L. Ryan, P. B. Bernard, A. Lau, J. P. Forder and others, “Effectiveness of PSD95 inhibitors in permanent and transient focal ischemia in the rat,” Stroke, vol. 39, p. 2544-2553, 2008.
[23] D. J. Cook, L. Teves and M. Tymianski, “Treatment of stroke with a PSD-95 inhibitor in the gyrencephalic primate brain,” Nature, vol. 483, p. 213-217, 2012.
[24] M. D. Hill, M. Goyal, B. K. Menon, R. G. Nogueira, R. A. McTaggart, A. M. Demchuk, A. Y. Poppe, B. H. Buck, T. S. Field, D. Dowlatshahi and others, “Efficacy and safety of nerinetide for the treatment of acute ischaemic stroke (ESCAPE-NA1): a multicentre, double-blind, randomised controlled trial,” The Lancet, vol. 395, p. 878-887, 2020.
[25] C. Peeters-Scholte, J. Koster, W. Veldhuis, E. van den Tweel, C. Zhu, N. Kops, K. Blomgren, D. Bär, S. van Buul-Offers, H. Hagberg and others, “Neuroprotection by selective nitric oxide synthase inhibition at 24 hours after perinatal hypoxia-ischemia,” Stroke, vol. 33, p. 2304-2310, 2002.
[26] E. R. v. d. Tweel, F. Van Bel, A. Kavelaars, C. M. P. C. D. Peeters-Scholte, J. Haumann, C. H. A. Nijboer, C. J. Heijnen and F. Groenendaal, “Long-term neuroprotection with 2-iminobiotin, an inhibitor of neuronal and inducible nitric oxide synthase, after cerebral hypoxia-ischemia in neonatal rats,” Journal of Cerebral Blood Flow & Metabolism, vol. 25, p. 67-74, 2005.
[27] C. H. A. Nijboer, F. Groenendaal, A. Kavelaars, H. H. Hagberg, F. Van Bel and C. J. Heijnen, “Gender-Specific Neuroprotection by 2-Iminobiotin after Hypoxia-Ischemia in the Neonatal Rat via a Nitric Oxide Independent Pathway,” Journal of Cerebral Blood Flow & Metabolism, vol. 27, p. 282-292, 2007.
[28] L. M. A. Favié, C. M. P. C. D. Peeters-Scholte, A. Bakker, H. Tjabbes, T. C. G. Egberts, F. van Bel, C. M. A. Rademaker, P. Vis and F. Groenendaal, “Pharmacokinetics and short-term safety of the selective NOS inhibitor 2-iminobiotin in asphyxiated neonates treated with therapeutic hypothermia,” Pediatric Research, vol. 87, p. 689-696, 2020.
[29] T. Biselele, J. Bambi, D. M. Betukumesu, Y. Ndiyo, G. Tabu, J. Kapinga, V. Bola, P. Makaya, H. Tjabbes, P. Vis and others, “A Phase IIa Clinical Trial of 2-Iminobiotin for the Treatment of Birth Asphyxia in DR Congo, a Low-Income Country,” Pediatric Drugs, vol. 22, p. 95-104, 2020.
[30] E.-J. van Hoogdalem, C. M. P. C. D. Peeters-Scholte, P. W. T. J. Leufkens, J. Hartstra, J. J. van Lier and L. G. J. de Leede, “First-in-Human Study of the Safety, Tolerability, Pharmacokinetics and -Preliminary Dynamics of Neuroprotectant 2-Iminobiotin in Healthy Subjects,” Current Clinical Pharmacology, vol. 15, p. 152-163, 10 2020.
[31] J. L. Saver, R. Chapot, R. Agid, A. E. Hassan, A. P. Jadhav, D. S. Liebeskind, K. Lobotesis, D. Meila, L. Meyer, G. Raphaeli and others, “Thrombectomy for Distal, Medium Vessel Occlusions: A Consensus Statement on Present Knowledge and Promising Directions,” Stroke, vol. 51, p. 2872-2884, 2020.
[32] E. N. El-Barhoun, S. R. Gledhill and A. G. Pitman, “Circle of Willis artery diameters on MR angiography: an Australian reference database,” Journal of Medical Imaging and Radiation Oncology, vol. 53, p. 248-260, 2009.
[33] J. Shatri, D. Bexheti, S. Bexheti, S. Kabashi, S. Krasniqi, I. Ahmetgjekaj and V. Zhjeqi, “Influence of gender and age on average dimensions of arteries forming the circle of Willis study by magnetic resonance angiography on Kosovo's population,” Open Access Macedonian Journal of Medical Sciences, vol. 5, p. 714, 2017.
[34] M. A. Stefani, F. L. Schneider, A. C. H. Marrone and A. G. Severino, “Influence of the gender on cerebral vascular diameters observed during the magnetic resonance angiographic examination of willis circle,” Brazilian archives of Biology and technology, vol. 56, p. 45-52, 2013.
[35] M. Waqas, A. T. Rai, K. Vakharia, F. Chin and A. H. Siddiqui, “Effect of definition and methods on estimates of prevalence of large vessel occlusion in acute ischemic stroke: a systematic review and meta-analysis,” Journal of neurointerventional surgery, vol. 12, p. 260-265, 2020.
[36] A. M. H. Sailer, J. P. Grutters, J. E. Wildberger, P. A. Hofman, J. T. Wilmink and W. H. van Zwam, “Cost-effectiveness of CTA, MRA and DSA in patients with non-traumatic subarachnoid haemorrhage,” Insights into imaging, vol. 4, p. 499-507, 2013.
[37] D. S. Liebeskind, S. Bracard, F. Guillemin, R. Jahan, T. G. Jovin, C. B. L. M. Majoie, P. J. Mitchell, A. van der Lugt, B. K. Menon, L. San Román and others, “eTICI reperfusion: defining success in endovascular stroke therapy,” Journal of neurointerventional surgery, vol. 11, p. 433-438, 2019.
[38] A. D. Rule, T. S. Larson, E. J. Bergstralh, J. M. Slezak, S. J. Jacobsen and F. G. Cosio, “Using serum creatinine to estimate glomerular filtration rate: accuracy in good health and in chronic kidney disease,” Annals of internal medicine, vol. 141, p. 929-937, 2004.
[39] K. Zitta, C. Peeters-Scholte, L. Sommer, M. Gruenewald, L. Hummitzsch, K. Parczany, M. Steinfath and M. Albrecht, “2-Iminobiotin superimposed on hypothermia protects human neuronal cells from hypoxia-induced cell damage: an in vitro study,” Frontiers in pharmacology, vol. 8, p. 971, 2018.
[40] L. K. Kwah and J. Diong, “National institutes of health stroke scale (nihss),” Journal of physiotherapy, 2014.
[41] J. M. Bamford, P. A. Sandercock, C. P. Warlow and J. Slattery, “Interobserver agreement for the assessment of handicap in stroke patients.,” Stroke, vol. 20, p. 828-828, 1989.
[42] Jansen I G H, Mulder M J H L, Goldhoorn R B. Endovascular treatment for acute ischaemic stroke in routine clinical practice: prospective, observational cohort study (MR CLEAN Registry) BMJ 2018; 360:k949.
[43] A. Ois, E. Cuadrado-Godia, J. Jiménez-Conde, M. Gomis, A. Rodríguez-Campello, J. E. MartínezRodríguez, E. Munteis and J. Roquer, “Early arterial study in the prediction of mortality after acute ischemic stroke,” Stroke, vol. 38, p. 2085-2089, 2007.
[44] Y. Lin, V. Schulze, M. Brockmeyer, C. Parco, A. Karathanos, Y. Heinen, M. Gliem, H.-P. Hartung, G. Antoch, S. Jander and others, “Endovascular thrombectomy as a means to improve survival in acute ischemic stroke: a meta-analysis,” JAMA neurology, vol. 76, p. 850-854, 2019.
[45] D.-E. Kim, W.-S. Ryu, D. Schellingerhout, H.-G. Jeong, P. Kim, S.-W. Jeong, M.-S. Park, K.-H. Choi, J.-T. Kim, B. J. Kim and others, “Estimation of acute infarct volume with reference maps: a simple visual tool for decision making in thrombectomy cases,” Journal of stroke, vol. 21, p. 69, 2019.
[46] Nasreddine Z S, Phillips N A, Bedirian V, Charbonneau S, Whitehead V, Collin I, et al. The Montreal cognitive assessment, MoCA: a brief screening tool for mild cognitive impairment. J Am Geriatr Soc. 2005; 53(4):695-9.
[47] Gaetani L, Blennow K, Calabresi P, Di Filippo M, Parnetti L, Zetterberg H. Neurofilament light chain as a biomarker in neurological disorders. J Neurol Neurosurg Psychiatry. 2019 August; 90(8): 870-881
[48] R. von Kummer, J. P. Broderick, B. C. V. Campbell, A. Demchuk, M. Goyal, M. D. Hill, K. M. Treurniet, C. B. L. M. Majoie, H. A. Marquering, M. V. Mazya and others, “The Heidelberg Bleeding Classification: classification of bleeding events after ischemic stroke and reperfusion therapy,” Stroke, vol. 46, p. 2981-2986, 2015.
[49] Emberson J, Lees K R, Lyden P, Blackwell L, Albers G, Bluhmki E, et al. Effect of treatment delay, age, and stroke severity on the effects of intravenous thrombolysis with alteplase for acute ischaemic stroke: a meta-analysis of individual patient data from randomised trials. The Lancet. 2014; 384(9958):1929-35.
[50] Wardlaw J M, Murray V, Berge E, del Zoppo G, Sandercock P, Lindley R L, et al. Recombinant tissue plasminogen activator for acute ischaemic stroke: an updated systematic review and meta-analysis. Lancet. 2012; 379(9834):2364-72
[51] Chandra R V, Leslie-Mazwi T M, Mehta B P, Derdeyn C P, Demchuk A M, Menon B K, et al. Does the use of IV tPA in the current era of rapid and predictable recanalization by mechanical embolectomy represent good value? Journal of NeuroInterventional Surgery. 2016; 8(5):443-6.
[52] Yang P, Zhang Y, Zhang L, et al. Endovascular Thrombectomy with or without Intravenous Alteplase in Acute Stroke. N Engl J Med. 2020; 382(21): 1981-1993.

1. 2-iminobiotin (2-IB) for use in the treatment of an individual suffering from acute ischemic stroke (AIS), wherein 2-IB is administered in combination with a fibrinolytic drug, in particular Alteplase.

2. 2-IB for use according to claim 1, wherein the acute ischemic stroke is due to vessel occlusion eligible for endovascular mechanical thrombectomy, preferably due to large, medium or distal vessel occlusion more preferably due to large or medium vessel occlusion, more preferably large vessel occlusion, most preferably large vessel occlusion in the anterior circulation.

3. 2-IB for use according to claim 1 or 2, wherein the acute ischemic stroke is due to symptomatic intracranial vessel occlusion, preferably an occlusion of the internal carotid artery (ICA), one of the first and second segments of the middle cerebral artery (MCA M1 or M2), the anterior cerebral artery, the vertebral artery, the basilar artery, or the proximal posterior cerebral artery, more preferably an occlusion in the anterior circulation, most preferably an occlusion of the internal carotid artery (ICA) or one of the first and second segments of the middle cerebral artery (MCA M1 or M2).

4. 2-IB for use according to any of the preceding claims, wherein the individual undergoes, is scheduled to undergo, or has undergone endovascular mechanical thrombectomy (EVT), preferably within 24 hours after stroke onset or last seen well.

5. 2-IB for use according to any one of the preceding claims, wherein the acute ischemic stroke is followed by reperfusion of ischemic brain tissue

6. 2-IB for use according to any one of the preceding claims, wherein 2-IB selectively inhibits neuronal nitric oxide synthase (nNOS), selectively inhibits inducible nitric oxide synthase (iNOS), or selectively inhibits nNOS and iNOS.

7. 2-IB for use according to any one of the preceding claims, wherein the administration of 2-IB to the individual is started within 24 hours after stroke onset or last seen well, preferably within 18 hours, more preferably within 12 hours, most preferably within 8 hours after stroke onset or last seen well.

8. 2-IB for use according to any one of the preceding claims, wherein the administration of 2-IB to the individual is started within 6 hours after the diagnosis AIS due to LVO, preferably after computed tomographic angiography (CTA), magnetic resonance angiography (MRA), or intra-arterial digital subtraction angiography (DSA).

9. 2-IB for use according to any one of the preceding claims, wherein administration of 2-IB to the individual is started within 2 hours, preferably within 1 hour, preferably within 15 minutes after the individual has undergone EVT and EVT is completed, preferably with successful reperfusion in the angiosuite, said successful reperfusion preferably exceeding eTICI grade 2b50, more preferably exceeding grade 2b67, more preferably exceeding grade 2b, most preferably reaching grade 3.

10. 2-IB for use according to any one of the preceding claims, wherein 2-IB is administered as a loading dose, and/or a continuous infusion for a duration of between 4-6 hours or for a duration of between 6-48 hours, preferably 12-36 hours, more preferably 20-28 hours, most preferably for about 24 hours.

11. 2-IB for use according to any one of the preceding claims, wherein 2-IB is administered in a loading dose of between 1.5-5 mg, preferably of between 2-3 mg, more preferably of between 2-2.5 mg, most preferably of about 2.25 mg

12. 2-IB for use according to any one of the preceding claims, wherein 2-IB is administered as a continuous infusion of between 0.25-3 mg/hour, preferably of between 0.5-2.25 mg/hour, preferably of between 0.75-1.5 mg/hour, most preferably of about 1 mg/hour

13. 2-IB for use according to claim 12, wherein during at least part of the continuous infusion, the 2-IB dose is adjusted based on the individual's estimated glomerular filtration rate (eGFR).

14. 2-IB for use according to any one of claims 11-13, wherein 2-IB is administered in a loading dose, followed by a continuous infusion, wherein in at least the last 12 hours, preferably at least the last 16 hours, of the continuous infusion the 2-IB dose is adjusted based on the individual's eGFR.

15. 2-IB for use according to any one of the preceding claims, wherein the treatment results in lower stroke severity, when measured using the National Institutes of Health Stroke Scale at 24 hours, at about 5-7 days after treatment, at hospital discharge, and/or at about 90 days after treatment.

16. 2-IB for use according to any one of the preceding claims, wherein the treatment results in lower score on the modified Rankin Scale (mRS) for the evaluation of neurological functional disability when assessed at hospital discharge, and/or about 90 days.

17. 2-IB for use according to any one of the preceding claims, wherein the treatment results in lower mortality rates when assessed at about 90 days.

18. 2-IB for use according to any one of the preceding claims, wherein the treatment results in smaller infarct volume when measured with MRI at 24-48 hours (or CT in case of contraindication for MRI)

19. 2-IB for use according to any one of the preceding claims, wherein the treatment results in less embolization in a new territory on angiography during EVT or infarction in new territory on 24-48 h CTA, MRA of DSA.

20. 2-IB for use according to any one of the preceding claims, wherein treatment results in less severe depressive disorder in the individual, when measured with the Structured Clinical Interview for Depression or the Depression Interview and Structured Hamilton test, assessed at 30-90 days after treatment.

21. 2-IB for use according to any one of the preceding claims, wherein treatment results in improved quality of life of the individual, when measured with the EQ-5D questionnaire and/or with the hospital anxiety and depression scale (HADS).

23. 2-IB for use according to any one of the preceding claims, wherein treatment results in improved cognitive function of the individual, when measured with the Montreal Cognitive Assessment (MoCA).

24. 2-IB for use according to any one of the preceding claims, wherein treatment results in lower neurofilaments levels in blood of the individual at 24-72 hours after AIS, at hospital discharge, and/or at 3 months after AIS.

25. 2-IB for use according to any one of the preceding claims, wherein the treatment results in a lower risk of postintervention intracranial hemorrhage on neuroimaging according to the Heidelberg Bleeding Classification within 24 hours of study drug administration.

26. 2-IB for use according to any one of the preceding claims, wherein 0.1 to 10 mg/kg/day of 2-IB, preferably 0.2 to 5 mg/kg/day of 2-IB more preferably 0.2 to 1 mg/kg/day of 2-IB is administered to the individual.

27. 2-IB for use according to any one of the preceding claims, wherein 2-IB is administered such that the area under the plasma concentration time curve from 0 to 4 h is between 100 ng·h/mL to 2000 ng·h/mL.

28. 2-IB for use according to any one of claims 1-27, wherein 2-IB is administered intravenously.

29. 2-IB for use according to any one of claims 1-27, wherein 2-IB is administered intraarterially.
